(19) United States
(12) Patent Application Publication
(10) Pub. No.: US 2006/0270675 A1
(43) Pub. Date: Nov. 30, 2006

(54) Inhibitors of cholesterol ester transfer protein

(76) Inventors: Robert D. Groneberg, Boulder, CO (US); Charles Todd Eary, Longmont, CO (US)

Correspondence Address: HOGAN & HARTSON LLP, ONE TABOR CENTER, 1200 17TH STREET, SUITE 1500, DENVER, CO, 80202, US

(21) Appl. No.: 11/372,473

(22) Filed: Mar. 9, 2006

Related U.S. Application Data
(60) Provisional application No. 60/660,219, filed on Mar. 10, 2005.

(51) Int. Cl. A61K 31/50

(52) U.S. Cl. 514/247; 514/249

(57) ABSTRACT

This invention relates to inhibitors of CETP, and methods for producing these inhibitors. The invention also provides pharmaceutical compositions comprising the inhibitors of the invention and methods of utilizing the inhibitors and pharmaceutical compositions in the treatment and prevention of various disorders mediated by CETP.

### CROSS-REFERENCE TO RELATED APPLICATIONS

This application claims priority to U.S. Provisional Application No. 60/660,219 filed Mar. 10, 2005, which is incorporated herein by reference in its entirety.

### BACKGROUND OF THE INVENTION

1. Field of the Invention

This invention relates to cholesteryl ester transfer protein (CETP) inhibitors, pharmaceutical compositions containing such inhibitors, and the use of such inhibitors to treat certain disease/conditions optionally in combination with certain therapeutic agents.

2. Description of the State of the Art

Atherosclerosis and its associated coronary artery disease (CAD) is the leading cause of mortality in the industrialized world. Despite attempts to modify secondary risk factors (smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

Risk for development of this condition has been shown to be strongly correlated with certain plasma lipid levels. While elevated LDL cholesterol may be the most recognized form of dyslipidemia, it is by no means the only significant lipid associated contributor to CHD. Low HDL cholesterol is also a known risk factor for CHD (Gordon, D. J., et al., “High-density Lipoprotein Cholesterol and Cardiovascular Disease”, Circulation, (1989), 79: 8-15).

Therapies to raise HDL cholesterol levels have been limited. HMG-CoA reductase inhibitors and fibrates only raise HDL cholesterol levels slightly, and while niacin can more significantly raise HDL cholesterol levels, side effects severely reduce its tolerability and compliance. Therefore, alternative therapies to raise HDL cholesterol are needed.

Among the many factors controlling plasma levels of these disease dependent principles, cholesteryl ester transfer protein (CETP) activity affects all three. Cholesterol ester transfer protein (CETP) is a 70,000 dalton glycoprotein present in the plasma of humans and other animal species. The role of CETP is to transfer cholesterol ester, triglyceride, and to a limited extent phospholipids, between plasma lipoprotein particles. The lipoprotein particles involved include high-density lipoprotein (HDL), low density lipoprotein (LDL), very low density lipoprotein (VLDL) and chylomicrons. This effect on lipoprotein profile is believed to be proatherogenic, especially in subjects whose lipid profile constitutes an increased risk for CHD. Since CETP is involved in the homeostasis of the plasma lipoprotein pool, its regulation by inhibition in the plasma compartment should allow for an altering of the circulating levels of these lipoproteins.

Clinical trials utilizing inhibitors of CETP have demonstrated the ability to raise circulating HDL cholesterol levels by this mechanism. One study employing a CETP inhibitor demonstrated a 34% increase in HDL cholesterol after 4 weeks using a 900 mg/day dose (Circulation, 2002, 105:2159). Evaluation of another CETP inhibitor showed after four weeks, at the highest dose, a 106% elevation in HDL cholesterol using a 120 mg dose twice daily (N. Engl. J. Med., 2004, 350:1505-15). Elevating plasma HDL cholesterol levels by inhibiting the activity of CETP may provide an anti-atherogenic benefit in humans. Although this has not yet been proven in humans, in rabbits, a CETP inhibitor was shown to be anti-athereogenic (Nature 2000, 406: 203-207).

### SUMMARY OF THE INVENTION

This invention provides cholesteryl ester transfer protein (CETP) inhibitors, methods to produce these compounds, and pharmaceutical compositions containing them for treating a CETP-mediated disorder or condition. Disorders and conditions that can be treated include, but are not limited to, cerebrovascular disease, coronary artery disease, hypertension, ventricular dysfunction, cardiac arrhythmia, pulmonary vascular disease, peripheral vascular disease, reno-vascular disease, renal disease, splanchnic vascular disease, vascular hemostatic disease, diabetes, inflammatory disease, autoimmune disorders and other systemic disease indications, immune function modulation, pulmonary disease, anti-oxidant disease, sexual dysfunction, cognitive dysfunction, schistosomiasis and cancer in a mammal. CETP inhibitors of the invention may be useful for the treatment of atherosclerosis, peripheral vascular disease and dyslipidemias, including hyperbetalipoproteinemia, hypoalphalipoproteinemia, hypercholesterolemia, familial hypercholesterolemia and hypertriglyceridemia.

In general, one aspect of the invention relates to CETP inhibitors of the general Formula I:

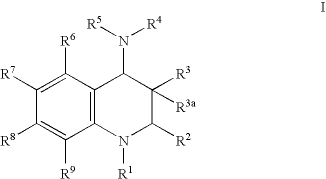

wherein

R¹ is Zₙ-(C═O)OR¹², Zₙ(C═O)Zₙ(C═O)OR¹², Zₙ-(C═O)NR¹²R¹³, Zₙ-NR¹²R¹³, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;

or R¹ is Zₙ-heterocycloalkyl substituted with a cycloalkyl group so as to form a bicyclic spirocycle, wherein said Zₙ-heterocycloalkyl is optionally substituted;

R², R³ and R^{3a} are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;

or R¹ and R² together with the atoms to which they are attached form a substituted or unsubstituted, saturated or partially unsaturated 5 or 6-membered heterocyclic ring;

R⁴ is Zₙ-Ar;

R⁵ is a fully saturated, partially unsaturated or fully unsaturated 4-7 membered heterocyclic ring having 1-4 atoms independently selected from O, N and S, wherein said heterocyclic ring may be substituted or unsubstituted;

R⁶, R⁷, R⁸, and R⁹ are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;

R¹² and R¹³ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or ZₙAr, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and ZₙAr may be substituted or unsubstituted;

Z is alkylene having from 1 to 4 carbons, or alkenylene or alkynylene each having from 2 to 4 carbons, wherein said alkylene, alkenylene, or alkynylene may be substituted or unsubstituted;

Ar is substituted or unsubstituted aryl or heteroaryl; and

n is 0, 1, 2, 3, or 4.

In another embodiment, the invention relates to CETP inhibitors of the general Formula Ia:

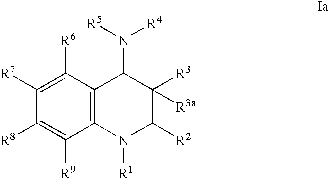

and resolved enantiomers, diastereomers, solvates, pharmaceutically acceptable salts and prodrugs thereof, wherein:

R¹ is Zₙ-(C═O)OR¹², Zₙ(C═O)Zₙ(C═O)OR¹², Zₙ-(C═O)NR¹²R¹³, Zₙ-NR¹²R¹³, alkyl, alkenyl, saturated or partially unsaturated Zₙ-cycloalkyl, saturated or partially unsaturated Zₙ-heterocyclyl or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹², and alkyl;

or R¹ is Zₙ-heterocyclyl substituted with a C₃-C₆ spirocyclic ring, wherein said Zₙ-heterocyclyl is optionally substituted with one or more groups independently selected from F, Cl, Br, I, OR¹², NR¹²R¹³, SR¹², and alkyl;

R², R³ and R^{3a} are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, alkenyl, alkynyl, Zₙ-cycloalkyl, Zₙ-heterocyclyl, or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;

or R¹ and R² together with the atoms to which they are attached form a substituted or unsubstituted, saturated or partially unsaturated 5 or 6-membered heterocyclic ring;

R⁴ is Zₙ-Ar;

R⁵ is a fully saturated, partially unsaturated or fully unsaturated 4-7 membered heterocyclic ring having 1-4 atoms independently selected from O, N and S, wherein said heterocyclic ring is optionally substituted with one or more groups independently selected from F, Cl, Br, I, Zₙ-OR¹², NR¹²R¹³, SR¹², Zₙ-C(═O)R¹², Zₙ-OP(═O)(OH)₂ and alkyl;

R⁶, R⁷, R⁸, and R⁹ are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, alkenyl, alkynyl, Zₙ-cycloalkyl, Zₙ-heterocyclyl, or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;

R¹² and R¹³ are independently H, alkyl, alkenyl, alkynyl, Zₙ-cycloalkyl, Zₙ-heterocyclyl, or ZₙAr, wherein said alkyl, alkenyl, alkynyl, Zₙ-cycloalkyl, Zₙ-heterocyclyl and ZₙAr are optionally substituted with one or more groups independently selected from F, Cl, Br, I, OR^{a}, NR^{a}R^{b}, SR^{a}, and alkyl;

Z is alkylene having from 1 to 4 carbons, or alkenylene or alkynylene each having from 2 to 4 carbon;

Ar is aryl or heteroaryl, wherein said aryl and heteroaryl are optionally substituted with one or more groups independently selected from F, Cl, Br, I, CF₃, OR^{a}, NR^{a}R^{b}, SR^{a}, and alkyl;

R^{a} and R^{b} are independently H, alkyl, alkenyl or alkynyl; and

n is 0 or 1.

In a further aspect the present invention provides a method of providing a CETP inhibitory effect comprising administering to a warm-blooded animal an effective amount of a compound of this invention, or a pharmaceutically acceptable salt or in vivo cleavable prodrug thereof, or a pharmaceutical composition comprising said compound.

In a further aspect the present invention provides methods of treating or preventing a CETP-mediated condition, comprising administering to a human or animal in need thereof a compound of this invention, or a pharmaceutically-acceptable salt or in vivo cleavable prodrug thereof, or a pharmaceutical composition comprising said compound, in an amount effective to treat or prevent said CETP-mediated condition.

The inventive compounds may be used advantageously in combination with other known therapeutic agents.

The invention also relates to pharmaceutical compositions comprising an effective amount of a compound of this invention, or a pharmaceutically acceptable prodrug, pharmaceutically active metabolite, or pharmaceutically acceptable salt thereof.

This invention also provides compounds of this invention for use as medicaments in the treatment of a CETP-mediated disease or disorder.

An additional aspect of the invention is the use of a compound of this invention for the preparation of a medicament for the treatment or prevention of a CETP-mediated disease or disorder,

This invention further provides kits for the treatment or prevention of a CETP-mediated disease or disorder, said kit comprising a compound of this invention, or a solvate, metabolite, or pharmaceutically acceptable salt or prodrug thereof, a container, and optionally a package insert or label indicating a treatment. The kits may further comprise a second compound or formulation comprising a second pharmaceutical agent useful for treating said disease or disorder.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF THE INVENTION

The inventive compounds of the present invention are useful for inhibiting CETP mediated events as described herein. In one embodiment, the method of treatment according to this invention results in a decrease in plasma small dense LDL, oxidized LDL, VLDL, apo(a) or Lp(a)) or an increase in pre-beta HDL, HDL-1, -2 and 3 particles.

In general, one aspect of the invention relates to CETP inhibitors of the general Formula I:

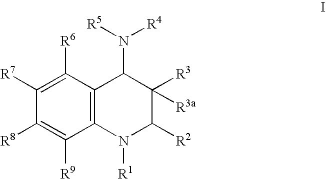

wherein

R¹ is Zₙ-(C═O)OR¹², Zₙ(C═O)Zₙ(C═O)OR¹², Zₙ-(C═O)NR¹²R¹³, Zₙ-NR¹²R¹³, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;

or R¹ is Zₙ-heterocycloalkyl substituted with a cycloalkyl group so as to form a bicyclic spirocycle, wherein said Zₙ-heterocycloalkyl is optionally substituted;

R², R³ and R^{3a} are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;

or R¹ and R² together with the atoms to which they are attached form a substituted or unsubstituted, saturated or partially unsaturated 5 or 6-membered heterocyclic ring;

R⁴ is Zₙ-Ar;

R⁵ is a fully saturated, partially unsaturated or fully unsaturated 4-7 membered heterocyclic ring having 1-4 atoms independently selected from O, N and S, wherein said heterocyclic ring may be substituted or unsubstituted;

R⁶, R⁷, R⁸, and R⁹ are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;

R¹² and R¹³ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or ZₙAr, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and ZₙAr may be substituted or unsubstituted;

Z is alkylene having from 1 to 4 carbons, or alkenylene or alkynylene each having from 2 to 4 carbons, wherein said alkylene, alkenylene, or alkynylene may be substituted or unsubstituted;

Ar is substituted or unsubstituted aryl or heteroaryl; and

n is 0, 1, 2, 3, or 4.

Another aspect of the invention provides compounds of the general Formula Ia:

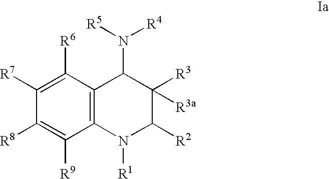

and resolved enantiomers, diastereomers, solvates, pharmaceutically acceptable salts and prodrugs thereof, wherein:

R¹ is Zₙ-(C═O)OR¹², Zₙ(C═O)Zₙ(C═O)OR¹², Zₙ-(C═O)NR¹²R¹³, Zₙ-NR¹²R¹³, alkyl, alkenyl, saturated or partially unsaturated Zₙ-cycloalkyl, saturated or partially unsaturated Zₙ-heterocyclyl or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;

or R¹ is Zₙ-heterocyclyl substituted with a C₃-C₆ spirocyclic ring, wherein said Zₙ-heterocyclyl is optionally substituted with one or more groups independently selected from F, Cl, Br, I, OR¹², NR¹²R¹³, SR¹² and alkyl;

R², R³ and R^{3a} are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, alkenyl, alkynyl, Zₙ-cycloalkyl, Zₙ-heterocyclyl, or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;

or R¹ and R² together with the atoms to which they are attached form a substituted or unsubstituted, saturated or partially unsaturated 5 or 6-membered heterocyclic ring;

R⁴ is Zₙ-Ar;

R⁵ is a fully saturated, partially unsaturated or fully unsaturated 4-7 membered heterocyclic ring having 1-4 atoms independently selected from O, N and S, wherein said heterocyclic ring is optionally substituted with one or more groups independently selected from F, Cl, Br, I, Zₙ-OR¹², NR¹²R¹³, SR¹², Zₙ-C(═O)R¹², Zₙ-OP(═O)(OH)₂ and alkyl;

R⁶, R⁷, R⁸, and R⁹ are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, alkenyl, alkynyl, Zₙ-cycloalkyl, Zₙ-heterocyclyl, or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;

R¹² and R¹³ are independently H, alkyl, alkenyl, alkynyl, Zₙ-cycloalkyl, Zₙ-heterocyclyl, or ZₙAr, wherein said alkyl, alkenyl, alkynyl, Zₙ-cycloalkyl, Zₙ-heterocyclyl and ZₙAr are optionally substituted with one or more groups independently selected from F, Cl, Br, I, OR^{a}, NR^{a}R_{b}, SR^{a}, and alkyl;

Z is alkylene having from 1 to 4 carbons, or alkenylene or alkynylene each having from 2 to 4 carbon;

Ar is aryl or heteroaryl, wherein said aryl and heteroaryl are optionally substituted with one or more groups independently selected from F, Cl, Br, I, CF₃, OR^{a}, NR^{a}R^{b}, SR^{a}, and alkyl;

R^{a} and R^{b} are independently H, alkyl, alkenyl or alkynyl; and

n is 0 or 1.

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like.

“Alkylene” means a linear or branched saturated divalent hydrocarbon radical of one to twelve carbon atoms, e.g., methylene, ethylene, propylene, 2-methylpropylene, pentylene, and the like.

The term “alkenyl” refers to linear or branched-chain monovalent hydrocarbon radical of two to twelve carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like.

The term “allyl” refers to a radical having the formula RC═CHCHR, wherein R is alkyl, alkenyl, alkynyl, cycloalkyl, heterocycloalkyl, aryl, heteroaryl, or any substituent as defined herein, wherein the allyl may be optionally substituted independently with one or more substituents described herein.

The term “alkenylene” refers to a linear or branched divalent hydrocarbon radical of two to twelve carbons containing at least one double bond, wherein the alkenylene radical may be optionally substituted independently with one or more substituents described herein. Examples include, but are not limited to, ethenylene, propenylene, and the like.

The term “alkynyl” refers to a linear or branched monovalent hydrocarbon radical of two to twelve carbon atoms with at least one site of unsaturation, i.e., a carbon-carbon, sp triple bond, wherein the alkynyl radical may be optionally substituted independently with one or more substituents described herein. Examples include, but are not limited to, ethynyl (—C≡CH), propynyl (propargyl, —CH₂C≡CH), and the like.

The term “alkynylene” to a linear or branched divalent hydrocarbon radical of two to twelve carbons containing at least one triple bond, wherein the alkynylene radical may be optionally substituted independently with one or more substituents described herein.

The term “heteroalkyl” refers to saturated linear or branched-chain monovalent hydrocarbon radical of one to twelve carbon atoms, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkyl radical may be optionally substituted independently with one or more substituents described herein. The term “heteroalkyl” encompasses alkoxy and heteroalkoxy radicals.

The term “heteroalkenyl” refers to linear or branched-chain monovalent hydrocarbon radical of two to twelve carbon atoms, containing at least one double bond, e.g., ethenyl, propenyl, and the like, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkenyl radical may be optionally substituted independently with one or more substituents described herein, and includes radicals having “cis” and “trans” orientations, or alternatively, “E” and “Z” orientations.

The term “heteroalkynyl” refers to a linear or branched monovalent hydrocarbon radical of two to twelve carbon atoms containing at least one triple bond. Examples include, but are not limited to, ethynyl, propynyl, and the like, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkynyl radical may be optionally substituted independently with one or more substituents described herein.

The term “heteroallyl” refers to radicals having the formula RC═CHCHR, wherein R is alkyl, alkenyl, alkynyl, cycloalkyl, heterocycloalkyl, aryl, heteroaryl, or any substituent as defined herein, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroallyl may be optionally substituted independently with one or more substituents described herein.

The terms “carbocycle”, “carbocyclyl”, “carbocyclic ring” and “cycloalkyl” refer to a monovalent non-aromatic, saturated or partially unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, and bicyclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Aryl includes bicyclic radicals comprising an aromatic ring fused to a saturated, partially unsaturated ring, or aromatic carbocyclic or heterocyclic ring. Typical aryl groups include, but are not limited to, radicals derived from benzene, substituted benzenes, naphthalene, anthracene, biphenyl, indenyl, indanyl, 1,2-dihydronapthalene, 1,2,3,4-tetrahydronapthyl, and the like.

The terms “heterocycle,” “hetercyclyl” and “heterocyclic ring” are used interchangeably herein and refer to a saturated or a partially unsaturated (i.e., having one or more double and/or triple bonds within the ring) carbocyclic radical of 3 to 20 ring atoms in which at least one ring atom is a heteroatom selected from nitrogen, oxygen and sulfur, the remaining ring atoms being C, where one or more ring atoms is optionally substituted independently with one or more substituents described below. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W.A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. The heterocyclyl may be a carbon radical or heteroatom radical. The term “heterocycle” includes heterocycloalkoxy. “Heterocyclyl” also includes radicals where heterocycle radicals are fused with a saturated, partially unsaturated ring, or aromatic carbocyclic or heterocyclic ring. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. Also included are heterocyclic rings substituted by one or more oxo (═O) moieties. Examples of a heterocyclic group wherein 2 ring carbon atoms are substituted with oxo moieties are pyrimidinonyl and 1,1-dioxo-thiomorpholinyl. The heterocycle groups herein are optionally substituted independently with one or more substituents described herein.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The heterocycle or heteroaryl groups may be C-attached or N-attached where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The term “halo” represents fluoro, chloro, bromo or iodo.

In general, the various moieties or functional groups of the compounds of this invention may be optionally substituted by one or more substituents. Examples of substituents suitable for purposes of this invention include, but are not limited to, halo, alkyl, alkenyl, alkynyl, Zₙ-cycloalkyl, Zₙ-heterocyclyl, Zₙ-OR^{a}, Zₙ-NO₂, Zₙ-CN, Zₙ-CO₂R^{a}, Zₙ-(C═O)R^{a}, Zₙ-O(C═O)R^{a}, Zₙ-O-alkyl, Zₙ-OAr, Zₙ-SR^{a}, Zₙ-SOR^{a}, Zₙ-SO₂R^{a}, Zₙ-S—Ar Zₙ-SOAr, Zₙ-SO₂Ar, Zₙ-Ar, Zₙ(C═O)NR^{a}R^{b}, Zₙ-NR^{a}R^{b}, Zₙ-PO₃H₂, Zₙ-SO₃H₂, amine protecting groups, alcohol protecting groups, sulfur protecting groups, and acid protecting groups, wherein Ar, Z, R^{a}, R^{b} and n are as defined herein.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and diastereomers, and mixtures, racemic or otherwise, thereof. Accordingly, this invention also includes all such isomers, including diastereomeric mixtures, pure diastereomers and pure enantiomers of the compounds of this invention. The term “enantiomer” refers to two stereoisomers of a compound which are non-superimposable mirror images of one another. The term “diastereomer” refers to a pair of optical isomers which are not mirror images of one another. Diastereomers have different physical properties, e.g. melting points, boiling points, spectral properties, and reactivities.

The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

In addition to compounds of Formulas I and Ia the invention also includes solvates, pharmaceutically acceptable prodrugs, and pharmaceutically acceptable salts of such compounds.

The phrase “pharmaceutically acceptable” indicates that the substance or composition is compatible chemically and/or toxicologically with the other ingredients comprising a formulation, and/or the mammal being treated therewith.

A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” can also be used to refer to a complex wherein the solvent molecule is water.

A “prodrug” is a compound that may be converted under physiological conditions or by solvolysis to the specified compound or to a salt of such compound. Prodrugs include compounds wherein an amino acid residue, or a polypeptide chain of two or more amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, cirtulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of this invention can be derivatized as an amide or alkyl ester. As another example, compounds of this invention comprising free hydroxy groups may be derivatized as prodrugs by converting the hydroxy group into a group such as, but not limited to, a phosphate ester, hemisuccinate, dimethylaminoacetate, or phosphoryloxymethyloxycarbonyl group, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group may be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., 1996, 39, 10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Free amines of compounds of this invention can also be derivatized as amides, sulfonamides or phosphonamides. All of these moieties may incorporate groups including, but not limited to, ether, amine and carboxylic acid functionalities. For example, a prodrug can be formed by the replacement of a hydrogen atom in the amine group with a group such as R-carbonyl, RO-carbonyl, NRR′-carbonyl, wherein R and R′ are each independently (C₁-C₁₀)alkyl, (C₃-C₇)cycloalkyl, or benzyl, or R-carbonyl is a natural α-aminoacyl or natural α-aminoacyl-natural α-aminoacyl, —C(OH)C(O)OY wherein Y is H, (C₁-C₆)alkyl or benzyl, —C(OY₀)Y₁ wherein Y₀ is (C₁-C₄)alkyl and Y₁ is (C₁-C₆)alkyl, carboxy(C₁-C₆)alkyl, amino(C₁-C₄)alkyl or mono-N- or di-N,N-(C₁-C₆)alkylaminoalkyl, or —C(Y₂)Y₃ wherein Y₂ is H or methyl and Y₃ is mono-N- or di-N,N-(C₁-C₆)alkylamino, morpholino, piperidin-1-yl or pyrrolidin-1-yl.

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

A “pharmaceutically acceptable salt,” unless otherwise indicated, includes salts that retain the biological effectiveness of the corresponding free acid or base of the specified compound and are not biologically or otherwise undesirable. A compound of the invention may possess a sufficiently acidic group, a sufficiently basic group, or both functional groups, and accordingly react with any of a number of inorganic or organic bases or acids to form a pharmaceutically acceptable salt. Examples of pharmaceutically acceptable salts include those salts prepared by reaction of the compounds of the present invention with a mineral or organic acid or an inorganic base, such salts including, but not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates. Since a single compound of the present invention may include more than one acidic or basic moiety, the compounds of the present invention may include mono, di or tri-salts in a single compound.

If the inventive compound is a base, the desired pharmaceutically acceptable salt may be prepared by any suitable method available in the art, for example, by treatment of the free base with an acidic compound, for example an inorganic acid such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid such as glucuronic acid or galacturonic acid, an alpha hydroxy acid such as citric acid or tartaric acid, an amino acid such as aspartic acid or glutamic acid, an aromatic acid such as benzoic acid or cinnamic acid, a sulfonic acid such as p-toluenesulfonic acid or ethanesulfonic acid, or the like.

If the inventive compound is an acid, the desired pharmaceutically acceptable salt may be prepared by any suitable method, for example, by treatment of the free acid with an inorganic or organic base. Examples of suitable inorganic salts include those formed with alkali and alkaline earth metals such as lithium, sodium, potassium, barium and calcium. Examples of suitable organic base salts include, for example, ammonium, dibenzylammonium, benzylammonium, 2-hydroxyethylammonium, bis(2-hydroxyethyl)ammonium, phenylethylbenzylamine, dibenzylethylenediamine, and the like salts. Other salts of acidic moieties may include, for example, those salts formed with procaine, quinine and N-methylglucosamine, plus salts formed with basic amino acids such as glycine, ornithine, histidine, phenylglycine, lysine and arginine.

The present invention also embraces isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified is contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Also falling within the scope of this invention are the in vivo metabolic products of compounds of this invention described herein. A “metabolite” is a pharmacologically active product produced through metabolism in the body of a specified compound or salt thereof. Such products may result, for example, from the oxidation, reduction, hydrolysis, amidation, deamidation, esterification, deesterification, enzymatic cleavage, and the like, of the administered compound. Accordingly, the invention includes metabolites of compounds of this invention, including compounds produced by a process comprising contacting a compound of this invention with a mammal for a period of time sufficient to yield a metabolic product thereof.

Metabolites are identified, for example, by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to a human, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolites, so long as they are not otherwise found in vivo, are useful in diagnostic assays for therapeutic dosing of the compounds of the invention.

The inventive compounds may be prepared using the reaction routes and synthesis schemes as described below, employing the techniques available in the art using starting materials that are readily available. However, it is to be understood that these schemes do not limit the invention and are only meant to suggest a method of practicing the invention. Persons skilled in the art will recognize that the chemical reactions described may be readily adapted to prepare a number of other CETP inhibitors of the invention, and alternative methods for preparing the compounds of this invention are deemed to be within the scope of this invention.

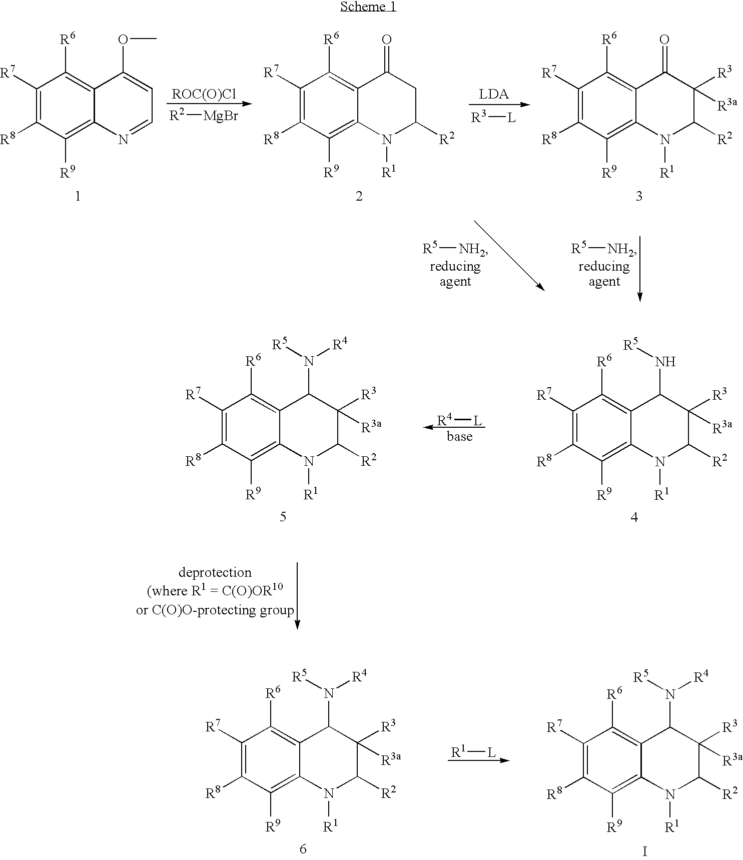

Scheme 1 illustrates an exemplary method for the preparation of compounds of Formula I. According to Scheme 1, a methoxy quinoline (1) is reacted with a Grignard reagent, (R²—MgBr) in the presence of a chloroformate to give a ketone (2). The newly formed R¹ group may optionally be incorporated into compound (2) as a protecting group such as tert-butyloxycarbonyl or benzyloxylcarbonyl, which may be exchanged for a different R¹ group later in the reaction scheme (see conversion of compound (6) to a compound of Formula I). The ketone intermediate (2) may be converted to the R⁵-amino analog (4) by a reductive amination using a reducing agent such as sodium cyanoborohydride with an amine R⁵—NH₂. Optionally, the ketone may be reacted with an electrophile to provide compound (3) comprising R³ and/or R^{3a} using a base, followed by reductive amination to provide compound (4). Functionalization of compound (4) to add R⁴ may be accomplished by alkylation using R⁴-L under basic conditions. Deprotection with either acidic or reducing conditions may be used to provide compound (6). Refunctionalization of a compound (6) using an alkylation or acylation provides the required R¹ group for the compound of Formula I. Compounds of Formula Ia can be made in a similar matter

Additional methods of preparing compounds of Formula I and Ia are described in the Examples.

The methods useful for the preparation of compounds of this invention as described herein may require protection of remote functionality (e.g., primary amine, secondary amine, and carboxyl groups). The need for such protection will vary depending on the nature of the remote functionality and the conditions of the preparation methods. The need for such protection is readily determined by one skilled in the art. The use of such protection/deprotection methods is also within the skill in the art. For a reference related to protecting groups and their use, see T. W. Greene, Protective Groups in Organic Synthesis, John Wiley & Sons, New York, 1991.

Representative examples of compounds of Formula I include

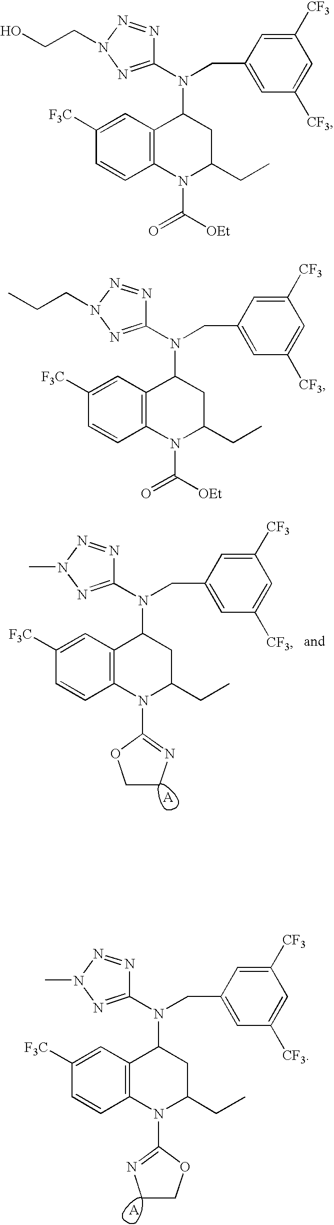

In one embodiment, ring A represents a C₃-C₆ spirocylic ring.

The compounds and pharmaceutical compositions of this invention are useful for treating disorders and conditions such as, but not limited to, cerebrovascular disease, coronary artery disease, hypertension, ventricular dysfunction, cardiac arrhythmia, pulmonary vascular disease, peripheral vascular disease, reno-vascular disease, renal disease, splanchnic vascular disease, vascular hemostatic disease, diabetes, inflammatory disease, autoimmune disorders and other systemic disease indications, immune function modulation, pulmonary disease, anti-oxidant disease, sexual dysfunction, cognitive dysfunction, schistosomiasis and cancer in a mammal, comprising administering to said mammal a therapeutically effective amount of a compound of this invention or a pharmaceutically acceptable salt thereof, in amounts that render the active agents effective in the treatment of said disorder or condition.

The term “cerebrovascular disease”, as used herein, includes, but is not limited to, ischemic attacks (e.g., transient), ischemic stroke (transient), acute stroke, cerebral apoplexy, hemorrhagic stroke, neurologic deficits post-stroke, first stroke, recurrent stroke, shortened recovery time after stroke and provision of thrombolytic therapy for stroke. Patient populations include, but are not limited to, patients with or without pre-existing stroke or coronary heart disease.

The term “coronary artery disease” includes, but is not limited to, atherosclerotic plaque (e.g., prevention, regression, stabilization), vulnerable plaque (e.g., prevention, regression, stabilization), vulnerable plaque area (reduction), arterial calcification (e.g., calcific aortic stenosis), increased coronary artery calcium score, dysfunctional vascular reactivity, vasodilation disorders, coronary artery spasm, first myocardial infarction, myocardia re-infarction, ischemic cardiomyopathy, stent restenosis, PTCA restenosis, arterial restenosis, coronary bypass graft restenosis, vascular bypass restenosis, decreased exercise treadmill time, angina pectoris/chest pain, unstable angina pectoris, exertional dyspnea, decreased exercise capacity, ischemia (reduce time to), silent ischemia (reduce time to), increased severity and frequency of ischemic symptoms, reperfusion after thrombolytic therapy for acute myocardial infarction.

The term “hypertension” includes, but is not limited to, lipid disorders with hypertension, systolic hypertension and diastolic hypertension.

The term “diabetes” includes, but is not limited to, type II diabetes, Syndrome X, Metabolic syndrome, lipid disorders associated with insulin resistance, non-insulin dependent diabetes, microvascular diabetic complications, reduced nerve conduction velocity, reduced or loss of vision, diabetic retinopathy, increased risk of amputation, decreased kidney function, kidney failure, metabolic syndrome, insulin resistance syndrome, pluri-metabolic syndrome, central adiposity (visceral) (upper body), diabetic dyslipidemia, decreased insulin sensitization, diabetic retinopathy/neuropathy, diabetic nephropathy/micro and macro angiopathy and micro/macro albuminuria, dyslipidemia, diabetic cardiomyopathy, diabetic gastroparesis, obesity, increased hemoglobin glycoslation, impaired renal and hepatic function.

The term “cognitive dysfunction” includes, but is not limited to, dementia secondary to atherosclerosis, transient cerebral ischemic attacks, neurodegeneration, neuronal deficient, and delayed onset or procession of Alzheimer's disease.

The term “ventricular dysfunction” includes, but is not limited to, systolic dysfunction, diastolic dysfunction, heart failure, congestive heart failure, dilated cardiomyopathy, idiopathic dilated cardiomyopathy, and non-dilated cardiomopathy.

The term “cardiac arrhythmia” includes, but is not limited to, atrial arrhythmias, supraventricular arrhythmias, ventricular arrhythmias and sudden death syndrome.

The term “pulmonary vascular disease” includes, but is not limited to, pulmonary hypertension and pulmonary embolism.

The term “peripheral vascular disease” includes, but is not limited to, peripheral vascular disease and claudication

The term “reno-vascular/renal disease” includes, but is not limited to, renal vascular diseases, renal hypertension and renal arterial stenosis.

The term “splanchnic vascular disease” includes, but is not limited to, ischemic bowel disease.

The term “vascular hemostatic disease” includes, but is not limited to, deep venous thrombosis, vaso-occlusive complications of sickle cell anemia, varicose veins, pulmonary embolism, transient ischemic attacks, embolic events, including stroke, in patients with mechanical heart valves, embolic events, including stroke, in patients with right or left ventricular assist devices, embolic events, including stroke, in patients with intra-aortic balloon pump support, embolic events, including stroke, in patients with artificial hearts, embolic events, including stroke, in patients with cardiomyopathy, embolic events, including stroke, in patients with atrial fibrillation or atrial flutter.

The terms “inflammatory disease,” “autoimmune disorders” and other systemic diseases include, but are not limited to, multiple sclerosis, rheumatoid arthritis, osteoarthritis, irritable bowel syndrome, irritable bowel disease, Crohn's disease, colitis, vasculitis, lupus erythematosis, sarcoidosis, amyloidosis, and apoptosis.

The term “pulmonary disease” includes, but is not limited to, pulmonary fibrosis, emphysema, obstructive lung disease, chronic hypoxic lung disease, antioxidant deficiencies, hyper-oxidant disorders and asthma.

The term “immune function disease” includes, but is not limited to, transplant vasculopathy, solid organ transplant rejection, transplant rejection, impaired toxin sequestration/removal, elevated levels of CXC chemokines, interleukins including interleukin-1, 6 and 8, neutrophil-activating protein-2 (NAP-2), melanoma growth stimulatory activity protein (MGSA), elevated levels of CC chemokines, RANTES, MIP-1 alpha and beta, MCP-1, -2, -3, -4, -5 Eotaxin-1, -2, -3, C-reactive protein including highly sensitive C-reactive protein and TNF-α.

The term “anti-oxidant disease”, as used herein, includes, but is not limited to, aging, mortality, apoptosis and increased oxidative stress

The term “sexual dysfunction” includes, but is not limited to, male sexual dysfunction, erectile dysfunction and female sexual dysfunction.

The term “cognitive dysfunction”, as used herein, is selected, but not limited to, the group consisting of dementia secondary to atherosclerosis, neurodegeneration, neuronal deficient, and delayed onset or procession of Alzheimer's disease.

Additionally, the compounds of this invention are also useful for neurodegenerative diseases such as Parkinson's, Huntington's disease, amyloid deposition and amylotrophic lateral sclerosis.

The term “cancer”, as used herein, is defined, but not limited to, resistance to chemotherapy, unregulated cell growth, hyperplasia (e.g., benign prostatic hyperplasia) and any of a number of abnormal multiplication or increase in the number of normal cells in normal arrangement in a tissue. The compounds and combinations included herein are also useful for cancer prevention.

The compounds of this invention are also useful for reducing global cardiovascular risk and global risk scores.

The compounds of this invention are also useful for modulation of plasma and or serum or tissue lipids or lipoproteins, such as HDL subtypes (e.g., increase, including pre-beta HDL, HDL-1, -2 and, 3 particles) as measured by precipitation or by apo-protein content, size, density, NMR profile, FPLC and charge and particle number and its constituents; and LDL subtypes (including LDL subtypes e.g., decreasing small dense LDL, oxidized LDL, VLDL, apo(a) and Lp(a)) as measured by precipitation, or by apo-protein content, size density, NMR profile, FPLC and charge; IDL and remnants (decrease); phospholipids (e.g., increase HDL phospholipids); apo-lipoproteins (increase A-I, A-II, A-IV, decrease total and LDL B-100, decrease B-48, modulate C-II, C-III, E, J); paraoxonase (increase, anti-oxidant effects, anti-inflammatory effects); decrease post-prandial (hyper)lipemia; decrease triglycerides, decrease non-HDL; elevate HDL in subjects with low HDL due to increased CETP mass or activity and optimize and increase ratios of HDL to LDL (e.g., greater than 0.25).

The term “treating,” as used herein, unless otherwise indicated, means reversing, alleviating, inhibiting the progress of, or preventing the disorder or condition to which such term applies, or one or more symptoms of such disorder or condition. The term “treatment,” as used herein, unless otherwise indicated, refers to the act of treating as “treating” is defined immediately above. “Treating” is intended to mean at least the mitigation of a disease condition in a mammal, such as a human, that is affected, at least in part, by CETP and includes, but is not limited to, preventing the disease condition from occurring in a mammal, particularly when the mammal is found to be predisposed to having the disease condition but has not yet been diagnosed as having it; modulating and/or inhibiting the disease condition; and/or alleviating the disease condition.

Therapeutically effective amounts of the compounds of the invention may be used to treat diseases mediated by modulation or regulation of protein kinases. An “effective amount” is intended to mean that amount of compound that, when administered to a mammal in need of such treatment, is sufficient to effect treatment for a disease mediated by CETP. Thus, for example, a therapeutically effective amount of a compound selected from of this invention or a salt, active metabolite or prodrug thereof, is a quantity sufficient to modulate, regulate, or inhibit the activity of one or more protein kinases such that a disease condition which is mediated by that activity is reduced or alleviated. The amount of a compound of this invention will correspond to such an amount will vary depending upon factors such as the particular compound, disease condition and its severity, the identity (e.g., weight) of the mammal in need of treatment, but can nevertheless be routinely determined by one skilled in the art.

Another aspect of this invention provides a compound of this invention for use in the treatment of the diseases or conditions described herein in a mammal, for example, a human, suffering from such disease or condition. Also provided is the use of a compound of this invention in the preparation of a medicament for the treatment of the diseases and conditions described herein in a warm-blooded animal, such as a mammal, for example a human, suffering from such disorder.

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition that comprises a compound of this invention in association with a pharmaceutically acceptable diluent or carrier.

The pharmaceutical compositions of the invention are formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutically effective amount of the compound to be administered will be governed by such considerations, and is the minimum amount necessary to prevent, ameliorate, or treat the disorder. The compound of the present invention is typically formulated into pharmaceutical dosage forms to provide an easily controllable dosage of the drug and to enable patient compliance with the prescribed regimen.

The composition for use herein is preferably sterile. In particular, formulations to be used for in vivo administration must be sterile. Such sterilization is readily accomplished, for example, by filtration through sterile filtration membranes. The compound ordinarily can be stored as a solid composition, a lyophilized formulation or as an aqueous solution.

Pharmaceutical formulations of the compounds of the present invention may be prepared for various routes and types of administration. For example, a compound of this invention having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, a milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment. The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more excipients.

The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The formulations may also include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the glucokinase inhibitors disclosed herein and, optionally, a chemotherapeutic agent) to a mammal. The components of the liposome are commonly arranged in a bilayer formation, similar to the lipid arrangement of biological membranes.

Sustained-release preparations of compounds of this invention may be prepared. Suitable examples of sustained-release preparations include semipermeable matrices of solid hydrophobic polymers containing a compound of this invention, which matrices are in the form of shaped articles, e.g., films, or microcapsules. Examples of sustained-release matrices include polyesters, hydrogels (for example, poly(2-hydroxyethylmethacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The pharmaceutical compositions of compounds of this invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The compositions of the invention may also be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder)

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

Suppository formulations may be prepared by mixing the active ingredient with a suitable non-irritating excipient that is solid at ordinary temperatures but liquid at the rectal temperature and will therefore melt in the rectum to release the drug. Suitable excipients include, for example, cocoa butter and polyethylene glycols. Formulations suitable for vaginal administration may be presented as pessaries, tampons, creams, gels, pastes, foams or spray formulations containing in addition to the active ingredient such carriers as are known in the art to be appropriate.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for transdermal administration may be in the form of those transdermal skin patches that are well known to those of ordinary skill in the art.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described herein.

The pharmaceutical composition (or formulation) for application may be packaged in a variety of ways depending upon the method used for administering the drug. For example, an article for distribution can include a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The formulations may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The amount of a compound of this invention that is combined with one or more excipients to produce a single dosage form will necessarily vary depending upon the subject treated, the severity of the disorder or condition, the rate of administration, the disposition of the compound and the discretion of the prescribing physician. In one embodiment, a suitable amount of a compound of this invention is administered to a mammal in need thereof. Administration in one embodiment occurs in an amount between about 0.001 mg/kg of body weight to about 60 mg/kg of body weight per day. In another embodiment, administration occurs in an amount between 0.5 mg/kg of body weight to about 40 mg/kg of body weight per day. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

The size of the dose for therapeutic or prophylactic purposes of a compound of this invention will naturally vary according to the nature and severity of the conditions, the age and sex of the animal or patient and the route of administration, according to well known principles of medicine.

In one aspect of this invention, the compounds of this invention or pharmaceutical salts or prodrugs thereof may be formulated into pharmaceutical compositions for administration to animals or humans to treat or prevent a CETP-mediated condition. The term “CETP-mediated condition” as used herein means any disease or other deleterious condition in which CETP is known to play a role.

In another embodiment of the invention, an article of manufacture, or “kit”, containing materials useful for the treatment of a CETP-mediated disorder or condition is provided. In one embodiment, the kit comprises a container comprising a compound of this invention. Suitable containers include, for example, bottles, vials, syringes, blister pack, etc. The container may be formed from a variety of materials such as glass or plastic. The container may hold a compound of this invention or a formulation thereof which is effective for treating the condition and may have a sterile access port (for example, the container may be an intravenous solution bag or a vial having a stopper pierceable by a hypodermic injection needle).

The kit may further comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. In one embodiment, the label or package inserts indicates that the composition comprising a compound of this invention can be used to treat a CETP-mediated disorder or condition. The label or package insert may also indicate that the composition can be used to treat other disorders.

In certain embodiments, the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to another embodiment, a kit may comprise (a) a first container with a compound of this invention contained therein; and (b) a second container with a second pharmaceutical formulation contained therein, wherein the second pharmaceutical formulation comprises a second compound useful for treating a CETP-mediated disorder or condition. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of this invention and, if present, the second pharmaceutical formulation. For example, if the kit comprises a first composition comprising a compound of this invention and a second pharmaceutical formulation, the kit may further comprise directions for the simultaneous, sequential or separate administration of the first and second pharmaceutical compositions to a patient in need thereof.

In certain other embodiments wherein the kit comprises a compound of this invention and a second therapeutic agent, the kit may comprise a container for containing the separate compounds such as a divided bottle or a divided foil packet, however, the separate compositions may also be contained within a single, undivided container. In certain embodiments, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other CETP inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was conducted on a Biotage system (Manufacturer: Dyax Corporation) having a silica gel column or on a silica SEP PAK® cartridge (Waters). ¹H NMR spectra were recorded on a Varian instrument operating at 400 MHz. ¹H NMR spectra were obtained as CDCl₃, using chloroform as the reference standard (7.25 ppm). When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

### Example 1

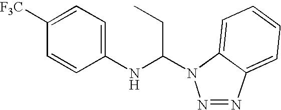

### (1-Benzotriazol-1-yl-propyl)-(4-trifluoromethyl-phenyl)-amine (7)

A one liter, single neck flask under nitrogen atmosphere was charged with benzotriazole (73.9 g, 621 mmol) and anhydrous toluene (900 mL). A solution of 4-(trifluoromethyl)aniline (100 g, 621 mmol) and toluene (50 mL) was added to the room temperature solution over 5 minutes. A solution of propionaldehyde (39.7 g, 683 mmol) and 50 mL of toluene was then added over 15 minutes. The reaction was stirred for 20 hours and then hexanes (400 mL) was added, and the slurry stirred an additional 20 minutes. The suspension was filtered and the filter cake washed with hexanes (2×100 mL) and then dried under high vacuum to yield compound 7 as a white powder (155 g, 78%).

### Example 2

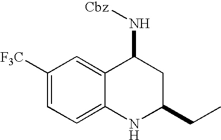

### cis-(2-ethyl-6-trifluoromethyl-1,2,3,4-tetrahydro-quinolin-4-yl)-carbamic acid benzyl ester (8)

To a solution of N-vinyl-carbamic acid benzyl ester (25.0 g, 141 mmol) in anhydrous toluene (500 mL) was added 7 (45.2 g, 141 mmol) and p-toluenesulfonic acid monohydrate (0.24 g, 1.41 mmol). The reaction was heated to 70° C. for 2 hours and then allowed to cool to room temperature. The reaction mixture was diluted with EtOAc (400 mL) and washed with 1N NaOH (200 mL), water (200 mL), and brine (200 mL). The organic phase was dried over Na₂SO₄, filtered and concentrated. Toluene (250 mL) was added, followed by 250 mL of hexanes to precipitate compound 8 as a white solid. The solid was filtered and dried under vacuum to provide 36.5 g (68%) of compound 8.

### Example 3

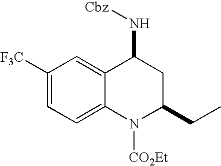

### cis-4-Benzyloxycarbonylamino-2-ethyl-6-trifluoromethyl-3,4-dihydro-2H-quinoline-1-carboxylic acid ethyl ester (9)

A 500 mL, single neck flask was charged with cis-(2-ethyl-6-trifluoromethyl-1,2,3,4-tetrahydro-quinolin-4-yl)-carbamic acid benzyl ester (8) (prepared according to Example 2; 30.0 g, 80.0 mmol), anhydrous dichloromethane (300 mL), and pyridine (9.00 g, 100 mmol). Ethyl chloroformate (10.0 g, 100 mmol) was added slowly over 30 minutes and then NaOH (100 mL, 1N) was added and the reaction stirred for 10 minutes. The separated organic layer was washed with water (100 mL), HCl (100 mL, 1N), sat. NaHCO3 (100 mL), brine (100 mL), then dried over Na₂SO₄ and filtered. The solids were rinsed with dichloromethane (100 mL). The filtrate was concentrated to dryness resulting in 33.8 g (90%) of compound 9 as a white powder.

### Example 4

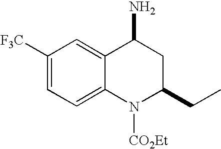

### cis-4-Amino-2-ethyl-6-trifluoromethyl-3,4-dihydro-2H-quinoline-1-carboxyxlic acid ethyl ester (10)

A 250 mL Parr hydrogenation flask was charged with cis-4-Benzyloxycarbonylamino-2-ethyl-6-trifluoromethyl-3,4-dihydro-2H-quinoline-1-carboxylic acid ethyl ester (9) (prepared according to Example 3; 9.15 g, 20.3 mmol), methanol (50 mL), and 10% Pd/C (50% water, 1.0 g). The solution was hydrogenated on a Parr shaker at 30 psi for 1 hour. The Pd/C was removed by filtration through Celite, rinsing the filter cake with methanol (2×100 mL). The resulting filtrate was concentrated to provide compound 10 (6.23 g, 97%) as a white powder.

### Example 5

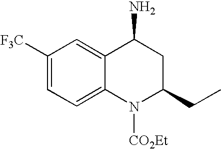

### (−) (2R,4S)-4-Amino-2-ethyl-6-trifluoromethyl-3,4-dihydro-2H-quinoline-1-carboxylic acid ethyl ester (11)

A 250 mL, single neck flask was charged with cis-4-Amino-2-ethyl-6-trifluoromethyl-3,4-dihydro-2H-quinoline-1-carboxyxlic acid ethyl ester (10) (prepared according to Example 4; 6.23 g, 19.7 mmol), (−) dibenzoyl-L-tartaric acid (7.06 g, 19.7 mmol) and ethanol (138 mL, 190 proof). The solution was stirred until all of the solids had dissolved. The stirrer was turned off and precipitation was allowed to progress for 24 hours at 5° C. (refrigerator). The solids were collected by filtration, rinsing with cold ethanol (20 mL). The solids were partitioned between dichloromethane (200 mL) and NaOH (200 mL, 1N). The organic layer was washed with NaOH (100 mL, 1N), brine (100 mL), then dried over Na₂SO₄ and filtered. The filtrate was concentrated to dryness resulting in 1.34 g (21%) of compound 11.

### Example 6

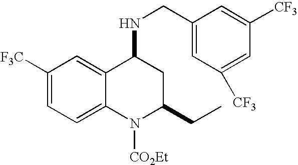

### (2R,4S)-ethyl 4-(3,5-bis(trifluoromethyl)benzylamino)-2-ethyl-6-(trifluoromethyl)-3,4-dihydroquinoline-1(2H)-carboxylate (12)

To a solution of (−) (2R,4S)-4-Amino-2-ethyl-6-trifluoromethyl-3,4-dihydro-2H-quinoline-1-carboxylic acid ethyl ester (11) (prepared according to Example 5; 365 mg, 1.15 mmol) in DCE (10 mL) was added 3,5-bis-trifluoromethyl benzaldehyde (293 mg, 1.21 mmol), acetic acid (0.066 mL, 1.15 mmol) and NaBH(OAc)₃ (489 mg, 2.31 mmol). The mixture was stirred at room temperature for 16 hours and then partitioned between saturated NaHCO₃ (25 mL) and EtOAc (25 mL). The organic layer was removed and the aqueous was extracted with EtOAc (2×25 mL). The combined organics were washed with brine and dried over MgSO₄. The solvent was removed under vacuum to produce compound 12 (590 mg, 94%) as a colorless oil, which solidified upon standing.

### Example 7

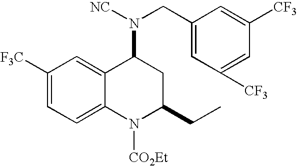

### (2R,4S)-ethyl 4-((3,5-bis(trifluoromethyl)benzyl)cyanamido)-2-ethyl-6-(trifluoromethyl)-3,4-dihydroquinoline-1(2H)-carboxylate (13)

Cyanogen bromide (185 mg, 1.75 mmol) was added to a suspension of (2R,4S)-ethyl 4-(3,5-bis(trifluoromethyl)benzylamino)-2-ethyl-6-(trifluoromethyl)-3,4-dihydroquinoline-1(2H)-carboxylate (12) (prepared according to Example 6; 790 mg, 1.46 mmol) and sodium carbonate (245 mg, 2.91 mmol) in EtOH at room temperature. After 4 hours the solids were removed via filtration and the filtrate was concentrated. The concentrate was purified via flash chromatography (100% hexanes to 10% EtOAc) to yield compound 13 (826 mg, 82%).

### Example 8

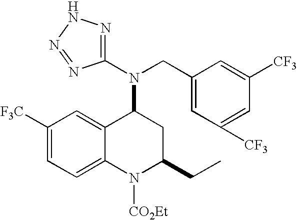

### (2R,4S)-ethyl 4-((3,5-bis(trifluoromethyl)benzyl)(2H-tetrazol-5-yl)amino)-2-ethyl-6-(trifluoromethyl)-3,4-dihydroquinoline-1(2H)-carboxylate (14)

To a solution of (2R,4S)-ethyl 4-((3,5-bis(trifluoromethyl)benzyl)cyanamido)-2-ethyl-6-(trifluoromethyl)-3,4-dihydroquinoline-1(2H)-carboxylate (13) (prepared according to Example 7; 675 mg, 1.19 mmol) in DMF (35 mL) was added ammonium chloride (318 mg, 5.95 mmol) and sodium azide (387 mg, 5.95 mmol). The mixture was heated to 60° C. for 12 hours. Upon cooling the reaction mixture was partitioned between EtOAc and water. The aqueous phase was removed and the organic layer was washed with brine (2×) and concentrated to yield compound 14 (543 mg, 75%). The crude material was used without purification.

### Example 9

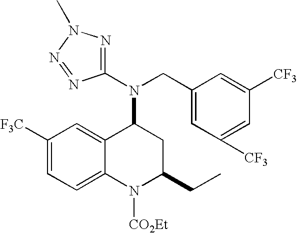

### (2R,4S)-Ethyl 4-((3,5-bis(trifluoromethyl)benzyl)(2-methyl-2H-tetrazol-5-yl)amino)-2-ethyl-6-(trifluoromethyl)-3,4-dihydroquinoline-1(2H)-carboxylate (15)

A solution of TMS-diazomethane (0.10 mL, 2.0 M) was slowly added to (2R,4S)-ethyl 4-((3,5-bis(trifluoromethyl)benzyl)(2H-tetrazol-5-yl)amino)-2-ethyl-6-(trifluoromethyl)-3,4-dihydroquinoline-1(2H)-carboxylate (14) (prepared according to Example 15; 59 mg, 0.10 mmol) in THF/MeOH (4:1). After 10 minutes the volatiles were removed and the concentrate was purified via preparative TLC (25% EtOAc/hexanes) to yield 43 mg (71%) of compound 15. ¹H NMR (400 MHz, CDCl₃) δ 7.79 (br s, 3H), 7.61 (d, 1H), 7.51 (d, 1H), 7.08 (s, 1H), 5.55-5.00 (br s, 2H), 4.75-4.45 (br m, 1H), 4.31-4.21 (m, 2H), 4.19 (s, 3H), 2.42-2.35 (m, 1H), 1.76-1.69 (m, 2H), 1.53-1.45 (m, 2H), 1.31 (t, 3H), 0.78 (t, 3H). C₂₆H₂₅F₉N₆O₂ MW=624.19, observed M+H=624.8.

### Example 10

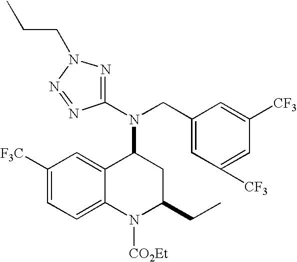

### (2R,4S)-ethyl 4-((3,5-bis(trifluoromethyl)benzyl)(2-propyl-2H-tetrazol-5-yl)amino)-2-ethyl-6-(trifluoromethyl)-3,4-dihydroquinoline-1(2H)-carboxylate (16)

Sodium hydride (2 mg, 0.081 mmol, 60%) was added to a solution of 4-((3,5-bis(trifluoromethyl)benzyl)(2H-tetrazol-5-yl)amino)-2-ethyl-6-(trifluoromethyl)-3,4-dihydroquinoline-1(2H)-carboxylate (14) (prepared according to Example 15; 41 mg, 0.067 mmol) in DMF (3 mL). After about 5 minutes 1-bromopropane (10 mg, 0.081 mmol) was added and the reaction was allowed to stir for 12 hours. The mixture was partitioned between EtOAc and water/brine. The aqueous was removed and the organic layer was washed with water (2×20 mL). The combined organics were dried and concentrated. The concentrate was purified via preparative TLC (10% EtOAc/hexanes) to yield compound 16 (20 mg, 45%). ¹H NMR (400 MHz, CDCl₃) δ 7.81(br s, 3H), 7.60 (d, 1H), 7.50 (d, 1H), 7.07 (s, 1H), 5.60-5.20 (br s, 1H), 5.06-5.02 (m, 1H), 4.80-4.28 (m, 3H), 4.32-4.17 (m, 2H), 2.43-2.36 (m, 1H), 1.99-1.91 (m, 2H), 1.76-1.70 (m, 2H), 1.53-1.44 (m, 2H), 1.31 (t, 3H), 0.90 (t, 3H), 0.79 (t, 3H). C₂₈H₂₉F₉N₆O₂ MW=652.5, observed 652.8.

### Example 11

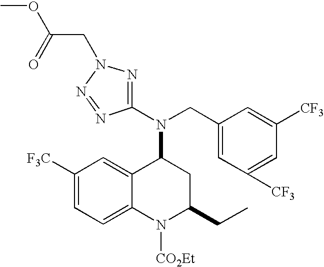

### (2R,4S)-ethyl 4-((3,5-bis(trifluoromethyl)benzyl)(2-(2-methoxy-2-oxoethyl)-2H-tetrazol-5-yl)amino)-2-ethyl-6-(trifluoromethyl)-3,4-dihydroquinoline-1(2H)-carboxylate (17)

Prepared according to the method of Example 10. ¹H NMR (400 MHz, CDCl₃) δ 7.80 (br s, 3H), 7.61 (d, 1H), 7.51 (d, 1H), 7.07 (s, 1H), 5.24 (s, 2H), 5.20-5.03 (m, 1H), 4.50-4.40 (m, 2H), 4.32-4.17 (m, 2H), 3.77 (s, 3H), 2.49-2.36 (m, 1H), 1.76-1.68 (m, 2H), 1.55-1.45 (m, 2H), 1.31 (t, 3H), 0.79 (t, 3H). C₂₈H₂₇F₉N₆O₄ MW=682.53, observed 682.8.

### Example 12

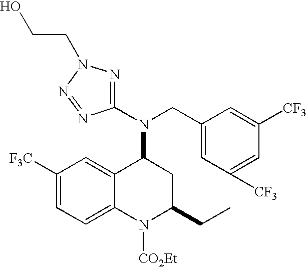

### (2R,4S)-Ethyl 4-((3,5-bis(trifluoromethyl)benzyl)(2-(2-hydroxyethyl)-2H-tetrazol-5-yl)amino)-2-ethyl-6-(trifluoromethyl)-3,4-dihydroquinoline-1(2H)-carboxylate (18)

A solution of LiBH₄ (0.07 mL, 2.0 M in THF) was added to (2R,4S)-ethyl 4-((3,5-bis(trifluoromethyl)benzyl)(2-(2-methoxy-2-oxoethyl)-2H-tetrazol-5-yl)amino)-2-ethyl-6-(trifluoromethyl)-3,4-dihydroquinoline-1(2H)-carboxylate (prepared according to Example 11; 107 mg, 0.16 mmol) in THF (5 mL). The mixture was heated to reflux for 1 hour. Upon cooling the mixture was partitioned between EtOAc and saturated NaHCO₃. The organic layer was removed, dried and concentrated. A portion (˜½) of the crude material was purified via preparative TLC (˜20% EtOAc/hexanes) to yield compound 18 (33 mg, 33%). ¹H NMR (400 MHz, CDCl₃) δ 7.81 (br s, 3H), 7.61 (d, 1H), 7.51 (d, 1H), 7.08 (s, 1H), 5.60-5.00 (m, 1H), 4.80-4.50 (m, 2H), 4.56-4.39 (m, 2H), 4.33-4.17 (m, 2H), 4.11-4.08 (m, 2H), 2.43-2.37 (m, 1H), 2.21-2.18 (t 1H), 1.78-1.68 (m, 2H), 1.55-1.46 (m, 2H), 1.31 (t, 3H), 0.79 (t, 3H). C₂₇H₂₇F₉N₆O₂ MW=654.53, observed 654.9.

### Example 13

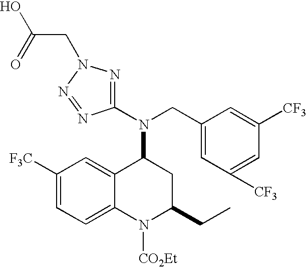

### 2-(5-((3,5-Bis(trifluoromethyl)benzyl)((2R,4S)-1-(ethoxycarbonyl)-2-ethyl-6-(trifluoromethyl)-1,2,3,4-tetrahydroquinolin-4-yl)amino)-2H-tetrazol-2-yl)acetic acid (19)

To a solution of (2R,4S)-ethyl 4-((3,5-bis(trifluoromethyl)benzyl)(2-(2-methoxy-2-oxoethyl)-2H-tetrazol-5-yl)amino)-2-ethyl-6-(trifluoromethyl)-3,4-dihydroquinoline-1(2H)-carboxylate (17) (prepared according to Example 11; 25 mg, 0.036 mmol) in THF/water (3:1, 4 mL) was added LiOH.H₂O. After 1 hour at room temperature the reaction was complete. The mixture was diluted in ether and washed with saturated NH₄Cl and then brine. The organic layer was dried (Na₂SO₄) and concentrated to yield compound 19 (24 mg, 99%). ¹H NMR (400 MHz, CDCl₃) δ 7.77 (br s, 3H), 7.56 (d, 1H), 7.46 (d, 1H), 7.04 (s, 1H), 5.20-5.00 (m, 3H), 4.45-4.35 (m, 2H), 4.30-4.10 (m, 2H), 2.40-2.30 (m, 1H), 1.75-1.65 (m, 2H), 1.45-1.41 (m, 2H), 1.26 (t, 3H), 0.75 (t, 3H). C₂₇H₂₅F₉N₆O₄ MW=668.51, observed 668.8.

### Example 14

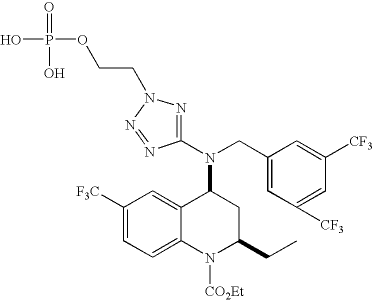

### (2R,4S)-Ethyl 4-((3,5-bis(trifluoromethyl)benzyl)(2-(2-(phosphonooxy)ethyl)-2H-tetrazol-5-yl)amino)-2-ethyl-6-(trifluoromethyl)-3,4-dihydroquinoline-1(2H)-carboxylate (20)

To a solution of (2R,4S)-Ethyl 4-((3,5-bis(trifluoromethyl)benzyl)(2-(2-hydroxyethyl)-2H-tetrazol-5-yl)amino)-2-ethyl-6-(trifluoromethyl)-3,4-dihydroquinoline-1(2H)-carboxylate (18) (prepared according to Example 12; 490 mg, 0.749 mmol) in dry DMF (10 mL) was added tetrazole (84.0 mg, 1.20 mmol) and di-t-butyl diisopropylphosphoramidite (355 μL, 1.12 mmol). After 14 hours at room temperature the reaction mixture was cooled to −78° C., and tert-butyl hydrogen peroxide (240 μL, 2.5 mmol) was added. The reaction was allowed to warm to room temperature and was stirred at room temperature for 16 hours. The reaction mixture was partitioned between ethyl acetate and aqueous sodium bicarbonate. The organic phase was washed with water three times followed by brine. The organic layer was dried (Na₂SO₄) and concentrated to yield the crude phosphate diester, which was purified by flash chromatography (5-20% EtOAc/hexanes) to provide the diester. The di-tert-butylester (480 mg, 0.567 mmol) was dissolved in a mixture of dichloromethane/trifluoroacetic acid (1:1, 5 ml) and was stirred at room temperature for 3 hours. The reaction mixture was concentrated and azeotroped with dichloromethane several times to provide the deprotected phosphate 20. ¹H NMR (400 MHz, CDCl₃) δ 7.90-7.78 (m, 5H), 7.61-7.50 (m, 2H), 7.09 (s, 1H), 5.09 (bd, 1H), 4.74-4.60 (m, 3H), 4.50-4.38 (m, 3H), 4.36-4.22 (m, 2H), 2.49-2.40 (m, 1H), 1.73-1.41 (m, 3H), 1.37-1.28 (m, 5H), 0.79 (t, 3H). C₂₇H₂₈F₉N₆O₆P MW=734.2, observed m/z 735 (M+H)⁺.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound including resolved enantiomers, diastereomers, solvates and pharmaceutically acceptable salts and prodrugs thereof, said compound having the Formula: 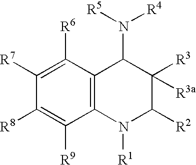
wherein
R¹ is Zₙ-(C═O)OR¹², Zₙ(C═O)Zₙ(C═O)OR¹², Zₙ-(C═O)NR¹²R¹³, Zₙ-NR¹²R¹³, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;
or R¹ is Zₙ-heterocycloalkyl substituted with a cycloalkyl group so as to form a bicyclic spirocycle, wherein said Zₙ-heterocycloalkyl is optionally substituted;
R², R³ and R^{3a} are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;
or R¹ and R² together with the atoms to which they are attached form a substituted or unsubstituted, saturated or partially unsaturated 5 or 6-membered heterocyclic ring;
R⁴ is Zₙ-Ar;
R⁵ is a fully saturated, partially unsaturated or fully unsaturated 4-7 membered heterocyclic ring having 1-4 atoms independently selected from O, N and S, wherein said heterocyclic ring may be substituted or unsubstituted;
R⁶, R⁷, R⁸, and R⁹ are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or Zₙ-Ar, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and Zₙ-Ar may be substituted or unsubstituted;
R¹² and R¹³ are independently H, alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl, or ZₙAr, wherein said alkyl, allyl, alkenyl, alkynyl, heteroalkyl, heteroallyl, heteroalkenyl, heteroalkynyl, alkoxy, heteroalkoxy, Zₙ-cycloalkyl, Zₙ-heterocycloalkyl and ZₙAr may be substituted or unsubstituted;
Z is alkylene having from 1 to 4 carbons, or alkenylene or alkynylene each having from 2 to 4 carbons, wherein said alkylene, alkenylene, or alkynylene may be substituted or unsubstituted;
Ar is substituted or unsubstituted aryl or heteroaryl; and
n is 0, 1, 2, 3, or 4.

2. The compound of claim 1, where R¹ is Zₙ-(C═O)OR¹².

3. The compound of claim 1, where R² is optionally substituted alkyl.

4. The compound of claim 1, where R⁷ is optionally substituted alkyl.

5. The compound of claim 4, where R⁷ is CF₃.

6. The compound of claim 1, where R¹ is a heteroaryl substituted with a C₃-C₆ spirocyclic ring.

7. The compound of claim 1, where R⁵ is 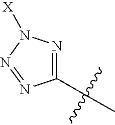
wherein X is H, alkyl, Zₙ-(C═O)OR¹², or Zₙ-OP(═O)(OH)₂, wherein said alkyl may be substituted or unsubstituted.

8. The compound of claim 1, which is

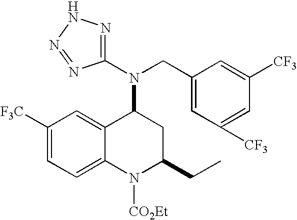

9. The compound of claim 1, which is

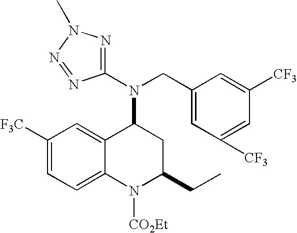

10. The compound of claim 1, which is

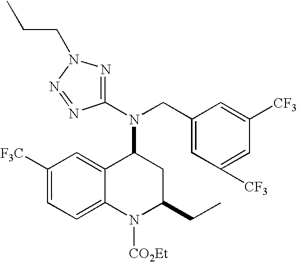

11. The compound of claim 1, which is

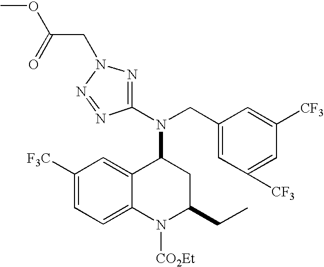

12. The compound of claim 1, which is

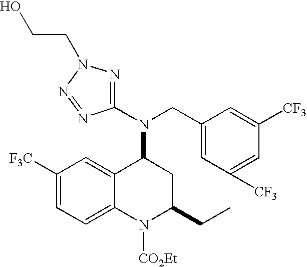

13. The compound of claim 1, which is

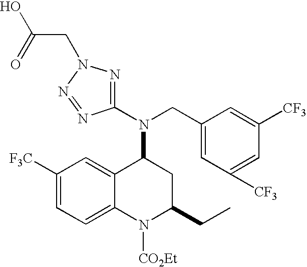

14. The compound of claim 1, which is

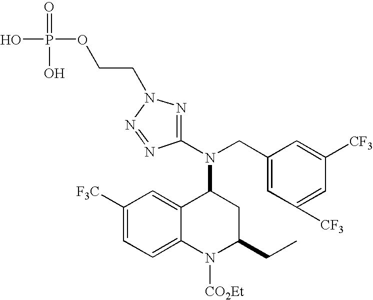

15. A compound having the Formula: 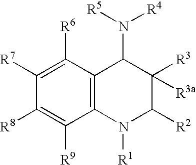
and resolved enantiomers, diastereomers, solvates, pharmaceutically acceptable salts and prodrugs thereof, wherein:
R¹ is Zₙ-(C═O)OR¹², Zₙ(C═O)Zₙ(C═O)OR¹², Zₙ-(C═O)NR¹²R¹³, Zₙ-NR¹²R¹³, alkyl, alkenyl, saturated or partially unsaturated Zₙ-cycloalkyl, saturated or partially unsaturated Zₙ-heterocyclyl or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;
or R¹ is Zₙ-heterocyclyl substituted with a C₃-C₆ spirocyclic ring, wherein said Zₙ-heterocyclyl is optionally substituted with one or more groups independently selected from F, Cl, Br, I, OR¹², NR¹²R¹³, SR¹² and alkyl;
R², R³ and R^{3a} are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, alkenyl, alkynyl, Zₙ-cycloalkyl, Zₙ-heterocyclyl, or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;
or R¹ and R² together with the atoms to which they are attached form a substituted or unsubstituted, saturated or partially unsaturated 5 or 6-membered heterocyclic ring;
R⁴ is Zₙ-Ar;
R⁵ is a fully saturated, partially unsaturated or fully unsaturated 4-7 membered heterocyclic ring having 1-4 atoms independently selected from O, N and S, wherein said heterocyclic ring is optionally substituted with one or more groups independently selected from F, Cl, Br, I, Zₙ-OR¹², NR¹²R¹³, SR¹², Zₙ-C(═O)R¹², Zₙ-OP(═O)(OH)₂ and alkyl;
R⁶, R⁷, R⁸, and R⁹ are independently H, OH, F, Cl, Br, I, CF₃, Zₙ-NR¹²R¹³, Zₙ-(C═O)NR¹²R¹³, Zₙ-SO₂R¹², Zₙ-SOR¹², Zₙ-SR¹², Zₙ-OR¹², Zₙ-(C═O)R¹², Zₙ-(C═O)OR¹², Zₙ-O—(C═O)R¹², alkyl, alkenyl, alkynyl, Zₙ-cycloalkyl, Zₙ-heterocyclyl, or Zₙ-Ar, wherein said alkyl, alkenyl, alkynyl, Zₙ-cycloalkyl, Zₙ-heterocyclyl and Zₙ-Ar are optionally substituted with one or more groups independently selected from F, Cl, Br, I, OR¹², NR¹²R¹³, SR¹² and alkyl;
R¹² and R¹³ are independently H, alkyl, alkenyl, alkynyl, Zₙ-cycloalkyl, Zₙ-heterocyclyl, or ZₙAr, wherein said alkyl, alkenyl, alkynyl, Zₙ-cycloalkyl, Zₙ-heterocyclyl and ZₙAr are optionally substituted with one or more groups independently selected from F, Cl, Br, I, OR^{a}, NR^{a}R^{b}, SR^{a}, and alkyll;
Z is alkylene having from 1 to 4 carbons, or alkenylene or alkynylene each having from 2 to 4 carbon;
Ar is aryl or heteroaryl, wherein said aryl and heteroaryl are optionally substituted with one or more groups independently selected from F, Cl, Br, I, CF₃, OR¹², NR¹²R¹³, SR¹² and alkyl;
R^{a} and R^{b} are independently H, alkyl, alkenyl or alkynyl; and
n is 0 or 1.

16. A pharmaceutical composition comprised of a compound of claim 1 and a pharmaceutically acceptable carrier.

17. A method of treating a CETP-mediated disease or disorder in a mammal, comprising administering a therapeutically effective amount of a compound of claim 1.

18. The use of a compound according to claim 1 in the manufacture of a medicament for the prophylactic or therapeutic treatment of a CETP-mediated disease or disorder in a mammal.

19. The use of a compound according to claim 1 for the treatment of a CETP-mediated disease or disorder in a mammal

20. A kit for treating a CETP-mediated condition, comprising:
a) a first pharmaceutical composition comprising a compound of claim 1; and
b) instructions for use.

21. The kit of claim 20, further comprising (c) a second pharmaceutical composition, wherein the second pharmaceutical composition comprises a second compound having CETP-inhibitory activity.

22. The kit of claim 21, further comprising instructions for the simultaneous, sequential or separate administration of said first and second pharmaceutical compositions to a patient in need thereof.

23. The kit of claim 22, wherein said first and second pharmaceutical compositions are contained in separate containers.

24. The kit of claim 22, wherein said first and second pharmaceutical compositions are contained in the same container.
